(19) United States
(12) Patent Application Publication
(10) Pub. No.: US 2016/0096005 A1
(43) Pub. Date: Apr. 7, 2016

(54) HYBRID INTRODUCER

(71) Applicant: Gyrus ACMI, Inc., d.b.a. Olympus Surgical Technologies America, Southborough, MA (US)

(72) Inventor: Shweta Bhola, Framingham, MA (US)

(21) Appl. No.: 14/505,586

(22) Filed: Oct. 3, 2014

(51) Int. Cl. A61M 25/09; A61B 17/221; A61B 1/012; A61B 1/00; A61B 1/018; A61B 6/12

(52) U.S. Cl. CPC A61M 25/09041; A61B 1/018; A61B 6/12; A61B 1/0125; A61B 1/00165; A61B 17/221; A61M 2025/09116; A61M 2025/09166; A61M 2025/09175

(57) ABSTRACT

A hybrid introducer and its use are provided. In one form, a method includes one or more of the following steps: feeding a guidewire through a hybrid introducer into a scope, the hybrid introducer including a proximal thumb advance portion and a distal tapered portion coupled to the proximal thumb advance portion; removing the proximal thumb advance portion; and feeding an accessory instrument through the distal tapered portion into the scope.

### FIELD

The present disclosure relates to a medical device. More specifically, the present disclosure relates to the use of a hybrid introducer for inserting a guidewire into an endoscope.

### BACKGROUND

The statements in this section merely provide background information related to the present disclosure and may or may not constitute prior art.

In certain medical procedures, a guidewire is inserted into an endoscope, and the tip of the guidewire is advanced to a particular location of interest in a patient's body. A sheath or catheter is slid over the guidewire, and the guidewire is subsequently withdrawn. After the guidewire is withdrawn, an accessory instrument is inserted through the sheath or catheter and advanced to the location of interest so that a physician can perform a desired medical procedure.

Among the literature that can pertain to this technology include the following patent documents and published patent applications: US 2007/0270892, U.S. Pat. No. 5,282,479, U.S. Pat. No. 5,263,938, and U.S. Pat. No. 5,980,492, all incorporated by reference for all purposes.

The guidewire is typically advanced or retracted with the use of a guidewire introducer. With certain introducers, there is a risk of the guidewire touching the ground and becoming contaminated before it is inserted into the patient. With other types of introducers, the advancement mechanism makes it difficult to feed other devices through the endoscope.

Accordingly, there is a need for effective and improved guidewire introducers.

### SUMMARY

The present disclosure provides an improved hybrid introducer and a method of using the hybrid introducer. In one aspect, the present disclosure provides a method including one or more of the following steps: feeding a guidewire through a hybrid introducer into a scope, the hybrid introducer including a proximal thumb advance portion and a distal tapered portion coupled to the proximal thumb advance portion; removing the proximal thumb advance portion; and feeding an accessory instrument through the distal tapered portion into the scope.

The invention may be further characterized by one or any combination of the features described herein, such as: removing the proximal thumb advance portion includes decoupling the proximal thumb advance portion from the distal tapered portion; the distal tapered portion is coupled to the proximal thumb advance portion with a snap fit mechanism or a screw mechanism; the scope is an endoscope; advancing a distal end of the guidewire to a desired location in a patient's body as the guidewire is being fed through the hybrid introducer; imaging the guidewire in the patient's body to determine if the distal end of the guidewire is at the desired location; imaging the guidewire includes generating an x-ray image of the guidewire in the patient's body; feeding a second endoscope over the guidewire; removing the proximal thumb advance portion of the hybrid introducer and feeding an accessory instrument through the distal tapered portion of the hybrid introducer into the second endoscope; removing the guidewire prior to feeding the accessory instrument through the distal tapered portion; the accessory instrument is an optical fiber; the accessory instrument is a retrieval basket; a distal end of the guidewire is a J-shaped end and the hybrid introducer has a passageway that straightens the J-shaped end.

Accordingly, pursuant to another aspect of the invention, there is contemplated a method including one or more of the following steps: feeding a guidewire through a hybrid introducer into a first endoscope, the hybrid introducer including a proximal thumb advance portion and a distal tapered portion coupled to the proximal thumb advance portion; advancing a distal end of the guidewire to a desired location in a patient's body and exchanging the first endoscope with a second endoscope; removing the proximal thumb advance portion from the hybrid introducer; attaching the distal tapered portion to the second endoscope; removing the guidewire; feeding an accessory instrument through the distal tapered portion into the endoscope; and advancing the accessory instrument through the second endoscope to the desired location.

Further features, advantages, and areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way. The components in the figures are not necessarily to scale, emphasis instead being placed upon illustrating the principles of the invention. Moreover, in the figures, like reference numerals designate corresponding parts throughout the views. In the drawings:

FIG. 1 is a side view of a hybrid introducer in accordance with the principles of the present invention;

FIG. 2 is an perspective view of the hybrid introducer with a guidewire contained in a protective tube;

FIG. 3 is a partial top view of the hybrid introducer;

FIG. 4 is a close-up expanded view of the hybrid introducer with a tapered portion that couples to a thumb advance portion with a screw mechanism in accordance with the principles of the present invention;

FIG. 5 is a close-up expand view of the hybrid introducer with the tapered portion that couples to the thumb advance portion with a snap fit mechanism in accordance with the principles of the present invention;

FIG. 6 is a flow diagram showing a process of using the hybrid introducer in accordance with the principles of the present invention; and

FIG. 7 is a close-up view of the hybrid introducer employed with an endoscope.

### DETAILED DESCRIPTION

The following description is merely exemplary in nature and is not intended to limit the present disclosure, application, or uses.

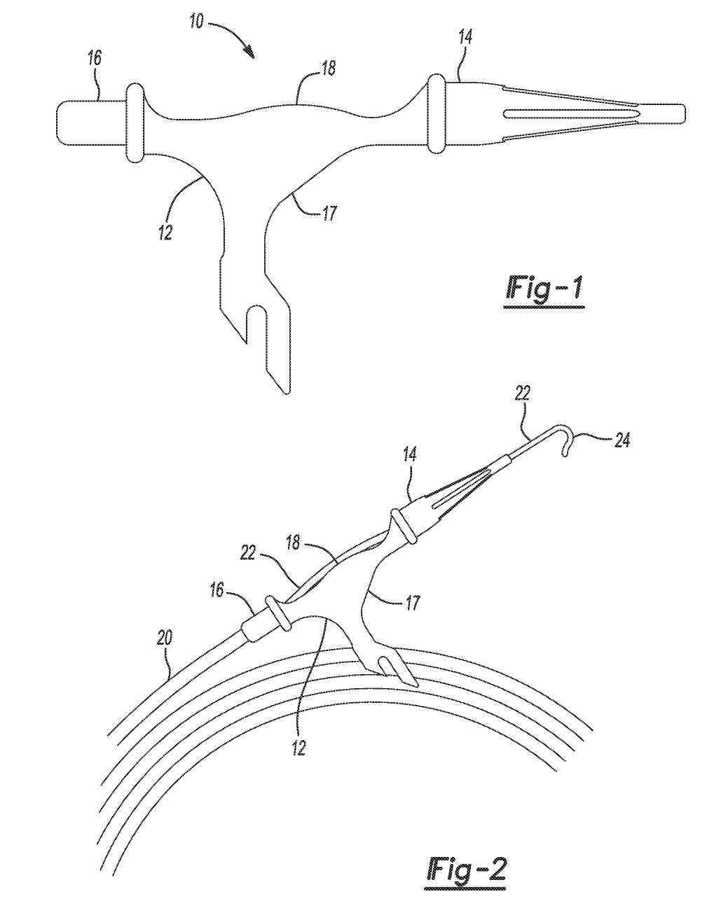

Referring now to the drawings, a hybrid introducer embodying the principles of the present invention is illustrated in FIG. 1 and designated at 10. The hybrid introducer 10 includes a proximal thumb advance portion 12 coupled to a distal tapered portion 14. The proximal advance portion 12 includes an extension 17 and a top region 18 that facilitates a user, such as, a physician, to hold and manipulate the hybrid introducer 10. Specifically, the user would typically wrap his/her index finger partially around the extension 17 while placing his/her digit, such as, a thumb, on the top region 18.

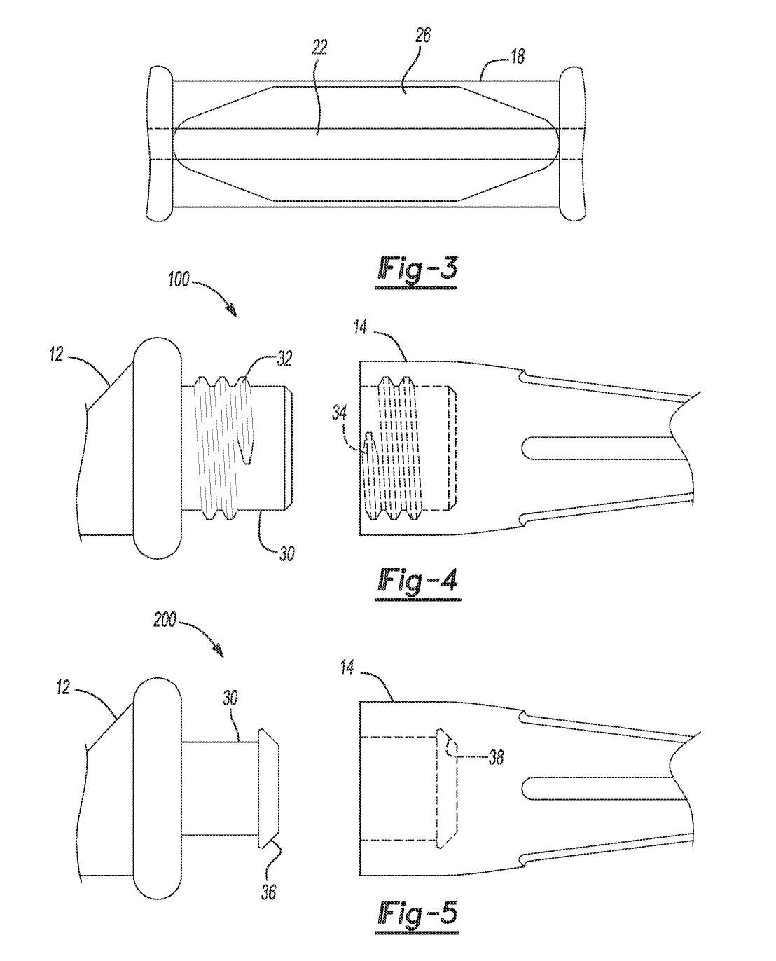

Referring further to FIG. 3, the top region 18 includes an opening 26 to provide access to a guidewire 22. Hence, the opening 26 allows the physician to use his/her thumb to advance or retract the guidewire in the hybrid introducer 10.

As shown in FIG. 2, a proximal region 16 of the hybrid introducer is affixed or coupled to a distal end of a protective tube 20, made of, for example, plastic. The guidewire 22 extends through the tube 20, the thumb advance portion 12 and the tapered portion 14. The distal end of the guidewire 22 may have a J-shaped distal end 24. As such, as the guidewire 22 is passed through the tube 20 and the tube advance portion 12, the guidewire 22 is straightened through the thumb advance portion 12. As the guidewire 22 is further advanced through the thumb advance portion 12, the guidewire 22 exits the tapered portion 14. As the guidewire 22 exits the tapered portion 14, the distal end of the guidewire 22 returns back to its preformed J-shaped end 24.

The distal tapered portion 14 can be coupled and de-coupled from the thumb advance portion 12 with any suitable mechanism. For example, as shown in FIG. 4, the thumb advance portion 12 can include a proximal region 30 with threads 32, and the tapered portion 14 can include matting threads 34. With such a configuration, the threads 32 and the matting threads 34 form a screw mechanism 100 that facilitates screwing and unscrewing the tapered potion 14 onto and from the thumb advance portion 12.

In another arrangement, the proximal region 30 includes a flared region 36 that couples to a bore region 38 of the tapered portion 14 to a form a snap fit mechanism 200 that facilitates coupling and de-coupling the tapered portion 14 to and from the thumb advance portion 12.

When the hybrid introducer 10 is in use, the hybrid introducer is typically employed in conjunction with a scope 400, as shown in FIG. 7. The scope can be, for example, an endoscope, such as a cystoscope, a colonoscope, or an ureteroscope. In the arrangement shown in FIG. 7, the scope 400 includes a raised portion 401 with a connector 402. The connector 402 may have outer threads that thread into inner threads of an adapter 404 to a seal 406 to the endoscope 400. Both the adapter 404 and the connector 402 have inner passageways so that the guidewire 22 can pass through the adapter 404 and the connector 402 into the endoscope 400 when the tapered portion 14 of the hybrid introducer 10 is inserted into the seal 406.

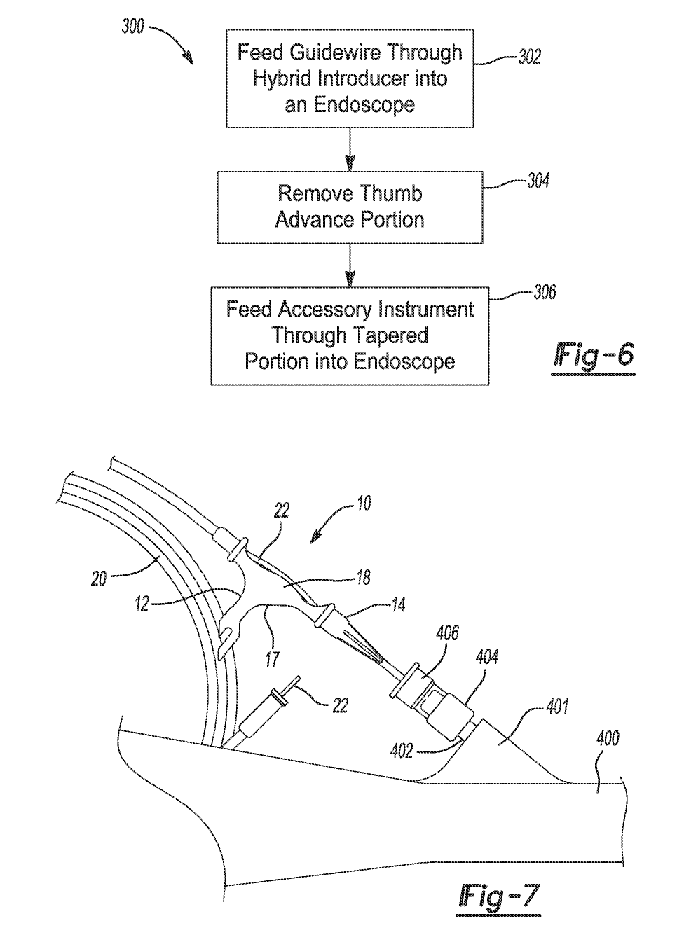

A further aspect of the disclosure provides a method 300 of using the hybrid introducer 10 in conjunction with the endoscope 400, as illustrated in the block diagram shown in FIG. 6. The method 300 includes a step 302 of feeding the guidewire 22 through the hybrid introducer 10 into the endoscope 400. The thumb advance portion 12 may be de-coupled from the tapered portion 14, with the screw mechanism 100 (FIG. 4) or the snap fit mechanism 200 (FIG. 5) described earlier, and removed in a step 304, and an accessory instrument may be fed through the tapered portion 14 into the endoscope 400.

In various arrangements, the physician can advance the distal end, for example, the J-shape end 24, of the guidewire 22 to a desired location in a patient's body as the guidewire 22 is being fed through the hybrid introducer 10. Imaging, such as, x-ray imaging, can be employed to determine if the distal end 24 of the guidewire 22 is at the desired location. The initial endoscope can be removed and a second endoscope, such as, for example, a cystoscope, a colonoscope, or an ureteroscope, can be fed over the guidewire 22. The thumb advance portion 12 can then be removed and an accessory instrument can be fed through the distal tapered portion 14 into the second endoscope.

With either the first or the second endoscope, the guidewire 22 may be removed prior to feeding the accessory instrument through the distal tapered portion 14. In various arrangements, the accessory instrument is an optical fiber or a retrieval basket.

The description of the invention is merely exemplary in nature and variations that do not depart from the gist of the invention are intended to be within the scope of the invention. Such variations are not to be regarded as a departure from the spirit and scope of the invention.

What is claimed is:

1. A method comprising:
feeding a guidewire through a hybrid introducer into a scope, the hybrid introducer including a proximal thumb advance portion and a distal tapered portion coupled to the proximal thumb advance portion;
removing the proximal thumb advance portion; and
feeding an accessory instrument through the distal tapered portion into the scope.

2. The method of claim 1 wherein removing the proximal thumb advance portion includes decoupling the proximal thumb advance portion from the distal tapered portion.

3. The method of claim 1 wherein the distal tapered portion is coupled to the proximal thumb advance portion with a snap fit mechanism.

4. The method of claim 1 wherein the distal tapered portion is coupled to the proximal thumb advance portion with a screw mechanism.

5. The method of claim 1 wherein the scope is an endoscope.

6. The method of claim 1 further comprising advancing a distal end of the guidewire to a desired location in a patient's body as the guidewire is being fed through the hybrid introducer.

7. The method of claim 6 further comprising imaging the guidewire in the patient's body to determine if the distal end of the guidewire is at the desired location.

8. The method of claim 7 wherein imaging the guidewire includes generating an x-ray image of the guidewire in the patient's body.

9. The method of claim 6 further comprising feeding a second endoscope over the guidewire.

10. The method of claim 9 further comprising removing the proximal thumb advance portion of the hybrid introducer and feeding an accessory instrument through the distal tapered portion of the hybrid introducer into the second endoscope.

11. The method of claim 1 further comprising removing the guidewire prior to feeding the accessory instrument through the distal tapered portion.

12. The method of claim 1 wherein the accessory instrument is an optical fiber.

13. The method of claim 1 wherein the accessory instrument is a retrieval basket.

14. The method of claim 1 wherein a distal end of the guidewire is a J-shaped end and the hybrid introducer has a passageway that straightens the J-shaped end.

15. A method comprising:
feeding a guidewire through a hybrid introducer into a first endoscope, the hybrid introducer including a proximal thumb advance portion and a distal tapered portion coupled to the proximal thumb advance portion;
advancing a distal end of the guidewire to a desired location in a patient's body and exchanging the first endoscope with a second endoscope;
removing the proximal thumb advance portion from the hybrid introducer;
attaching the distal tapered portion to the second endoscope;
removing the guidewire;
feeding an accessory instrument through the distal tapered portion into the endoscope; and
advancing the accessory instrument through the second endoscope to the desired location.
